(19) United States
(12) Patent Application Publication
(10) Pub. No.: US 2024/0420819 A1
(43) Pub. Date: Dec. 19, 2024

(54) Machine-Learning Based Motion Analysis and Training Method and System

(71) Applicants: Gyanna Gao, Gainesville, FL (US); Yang Gao, Gainesville, FL (US); Feihong Wang, Gainesville, FL (US); Hao-Yu Liao, Gainesville, FL (US); Peng Liu, Lebanon, NH (US)

(72) Inventors: Gyanna Gao, Gainesville, FL (US); Yang Gao, Gainesville, FL (US); Feihong Wang, Gainesville, FL (US); Hao-Yu Liao, Gainesville, FL (US); Peng Liu, Lebanon, NH (US)

(21) Appl. No.: 18/519,196

(22) Filed: Nov. 27, 2023

(51) Int. Cl. G16H 20/30; G06N 20/10

(52) U.S. Cl. CPC G16H 20/30; G06N 20/10

(57) ABSTRACT

A machine-learning (ML) based motion analysis and training method and systems are provided. The method includes acquiring data of measurements of kinematics of a user performing a task with his/her hands or arms; analyzing the data of measurements based on a machine-learning (ML) model to evaluate patterns of hand/arm movements of the user; and providing feedback and/or advice based on results of the analysis for improvement of skills of the hand/arm motions of the user. The analyzing the data of measurements includes data preprocessing for preparing raw data for analysis, feature engineering for creating relevant features from the raw data for training the ML model, model training for training the ML model based on the preprocessed data, model evaluating for assessing performance of the trained model based on predetermined evaluation metrics, hyperparameter tuning for fine-tuning the hyperparameters of the ML model to improve its performance, model validating, and model testing.

### BACKGROUND OF THE INVENTION

Numerous studies have demonstrated the manifold benefits of tennis for teenagers, encompassing enhancements in bone health, reduction in body fat percentage, and an overall boost in physical well-being (Babette, Staal, Marks, Miller, & Milley, 2007). Beyond its physiological advantages, tennis also plays a pivotal role in fostering character development among adolescents, thereby conferring a comprehensive array of positive outcomes.

However, many teens from low-income families cannot afford this sport. Solomon (2020) found that children from low-income households are much less likely to participate in tennis than children from high-income families, because of the cost that many economically disadvantaged families cannot afford. A main component of the cost is associated with coaching.

Nonetheless, a significant barrier arises as a great number of adolescents hailing from low-income households find themselves unable to embrace the realm of this sport. According to Solomon's extensive study in 2020, the inclination to engage in tennis among youngsters from less privileged backgrounds markedly diminishes in comparison to their counterparts from more affluent families. The disparity can be attributed to the substantial financial constraints borne by economically disadvantaged households, rendering them incapable of pursuing tennis. A main aspect of this financial restraint is intricately linked with the expenses associated with coaching services.

Traditionally, attaining proficiency in tennis requires the guidance of an expert coach or trainer. The acquisition and mastery of specialized tennis movement skills demand a substantial investment of time and effort. Many of these intricate movement skills fall under the umbrella of complex movements, which often feel unnatural and necessitate the adaptation of inherent functions to meet specific task demands. Familiarity with the external components of the task is also vital.

Typically, these movements are cultivated through a process of trial and error, wherein the focus remains on achieving desired outcomes. The journey towards becoming an accomplished amateur tennis player, eligible for participation in high school varsity teams, mandates thousands of hours dedicated to systematic and concentrated training to attain a commendable level of tennis prowess.

Throughout the training process, the feedback loop for players is rather limited, lacking substantial signals for correction and improvement. This void is especially prominent for underprivileged youth tennis players who lack access to coaching resources. Consequently, these players rely heavily on self-observation and repetitive drills to refine their movement skills. However, the rapid pace at which these movements unfold often makes them challenging to perceive. The complexity of these motions compounds the issue, involving multiple dimensions. For instance, the trajectory of a tennis racket is defined by the combination of three translational and rotational variables—essentially six degrees of freedom.

Furthermore, athletes often lack explicit awareness regarding the nuances of their motion execution. Consequently, their personal senses and judgments are frequently imprecise and unreliable. This underlines the intricate nature of tennis skill development and the significant role that expert guidance and targeted training play in nurturing truly proficient players.

Several self-training systems, such as ZEPP® tennis swing analyzer (available from Zepp US, Inc.) and the SMART TENNIS SENSOR (available from Sony), have been developed to analyze the performance of tennis players. However, these systems often require the expertise of a trained tennis analyst within a controlled environment, employing an array of video or infrared cameras strategically placed around the tennis court. In this setup, the athlete under scrutiny is affixed with markers at key points on their body. Subsequently, data is meticulously gathered and scrutinized by a tennis analyst, who then furnishes feedback to the athlete.

There are three primary issues associated with the existing self-training systems. The foremost problem is the limited availability due to the intricate nature of this data collection system, as wells the specialized hardware such as customized Inertial Measurement Units (IMUs) used by the existing self-training systems. Another significant problem is the high cost associated with most existing devices and software solutions. These offerings are often tailored for professionals and come with a hefty price tag. Further, these existing solutions are designed with professional tennis players in mind and thus suffer from complexity, rendering them challenging to use, especially for beginners and youth.

In order to enhance the accessibility of tennis for youngsters of low-income families, it is imperative to develop an application that can operate on widely used and inexpensive electronic devices such as smartphones, such that underprivileged children can be empowered to independently cultivate and refine their tennis abilities, without the substantial financial commitments tied to hiring a personal coach or enrolling in a tennis clinic.

### BRIEF SUMMARY OF THE INVENTION

There continues to be a need in the art for improved designs and techniques for tennis skill analysis and training system.

According to an embodiment of the subject invention, a machine-learning (ML) based motion analysis and training method comprises acquiring data of measurements of kinematics of a user performing a task with his/her hand or arm; analyzing the data of measurements based on a machine-learning (ML) model to evaluate patterns of band/arm movements of the user: and providing feedback and/or advice based on results of the analysis for improvement of skills of the hand/arm motions of the user. The acquiring data of measurements comprises measuring and collecting data of kinematic and/or temporal features of the movements through inertial measurements. The providing feedback and/or advice comprises providing instructions/feedback to the user based on the results of the analysis for the user to achieve desired skills of the hand/arm motions and displaying the instructions/feedback to the user in forms of text, audio, images, or videos. Moreover, the analyzing the data of measurements comprises data preprocessing configured to prepare raw data for analysis, feature engineering configured to create relevant features from the raw data for training the ML model, model training configured to train the ML model based on the preprocessed data, model evaluating configured to assess performance of the trained model based on predetermined evaluation metrics, hyperparameter tuning configured to fine-tune the hyperparameters of the ML model to improve its performance, model validating, and model testing. The data preprocessing comprises one or some or all of the steps including data cleaning, handling missing values, data transforming, and feature extracting. The feature engineering comprises one or some or all of the steps including dimensionality reduction, scaling, encoding categorical variables, and creating new features. The model training comprises feeding the data into the selected model and adjusting the model parameters to learn patterns from the data. In the step of model evaluating, the evaluation metrics comprise accuracy, precision, recall, F1-score, or area under the ROC curve (AUC-ROC). The hyperparameters of the step of hyperparameter tuning are settings that are not learned during the model training, including one or more of learning rate, regularization strength, and a number of hidden layers in a neural network. The data are split into a training set, a validation set, and a testing set to evaluate the model's performance on unseen data. The model training is based on training a support vector machine (SVM) model with the preprocessed data. In addition, the feature engineering comprises automatic segmentation by determining thresholds of the data. The automatic segmentation is performed by applying ruptures library for detecting changepoints of the data. The applying the ruptures library comprises applying a Pelt method or a Window method.

In certain embodiments of the subject invention, a computer program product comprising a non-transitory computer-executable storage device having computer readable program instructions embodied thereon that when executed by a computer cause the computer to perform a machine-learning (ML) based motion analysis and training method is provided. The computer-executable program instruction comprises acquiring data of measurements of kinematics of a user performing a task with his/her hand or arm; analyzing the data of measurements based on a machine-learning (ML) model to evaluate patterns of hand/arm movements of the user; and providing feedback and/or advice based on results of the analysis for improvement of skills of the hand/arm motions of the user.

In another embodiment of the subject invention, a machine-learning (ML) based motion analysis and training system comprises a data acquisition module configured for collecting data of measurement of kinematics of a user; a skill analysis module configured for wirelessly communicating with the data acquisition module and analyzing the data collected based on a machine-learning (ML) model to evaluate patterns of hand/arm movements of the user; and an instruction module configured for providing feedback and/or advice for improvement of skills of the hand/arm motions of the user. The skill analysis module is configured to train a support vector machine (SVM) model with the data of collected. Moreover, the skill analysis module is configured to preprocess data to prepare raw data for analysis. Further, the skill analysis module is configured to perform feature engineering to create relevant features from the raw data for training the ML model.

### BRIEF DESCRIPTION OF THE DRAWINGS

FIG. 1 is a schematic diagram illustrating hierarchy of tennis strokes including categories, types, classes, phases, and segments of phases, according to an embodiment of the subject invention.

FIG. 2 is a schematic representation illustrating a tennis player playing tennis strokes, according to an embodiment of the subject invention.

FIGS. 3A-3E are schematic diagrams illustrating the phases of strokes of tennis players of different skill levels, according to an embodiment of the subject invention.

FIG. 4 is a block diagram showing the overall structure of the machine-learning based motion analysis and training method and system, according to an embodiment of the subject invention.

FIG. 5A is a schematic diagram illustrating a linear coordinate system of Inertial Measurement Units (IMUs), according to an embodiment of the subject invention.

FIG. 5B is a schematic diagram illustrating an angular coordinate system of Inertial Measurement Units (IMUs), according to an embodiment of the subject invention.

FIG. 5C is an image showing an Apple Watch™ used for data collection, according to an embodiment of the subject invention.

FIG. 5D shows exemplary raw data collected by the IMU of the Apple Watch™. according to an embodiment of the subject invention.

FIG. 6 is a block diagram showing a skill analysis module of the machine-learning based motion analysis and training method and system, according to an embodiment of the subject invention.

FIG. 7 is a flow chart showing a data analysis unit of the skill analysis module of the machine-learning based motion analysis and training method and system, according to an embodiment of the subject invention.

FIG. 8 is a flow chart showing the implementations of skill level identification by the skill analysis module of the machine-learning based motion analysis and training method and system, according to an embodiment of the subject invention.

FIGS. 9A-9I are plot diagrams showing measurement results of the skill level identification of tennis players of different skill levels by the machine-learning based motion analysis and training method and system, according to an embodiment of the subject invention.

FIG. 10 is a schematic diagram illustrating transitions between different phases with transition thresholds, according to an embodiment of the subject invention.

FIG. 11 is a flow chart showing an implementation of the automatic phase identification process performed by the skill analysis module of the machine-learning based motion analysis and training method and system, according to an embodiment of the subject invention.

FIG. 12 is a flow chart showing another implementation of the automatic phase identification process performed by the skill analysis module of the machine-learning based motion analysis and training method and system, according to an embodiment of the subject invention.

FIG. 13 is a schematic representation illustrating a golf player practicing golf swings, according to an embodiment of the subject invention.

FIG. 14 is an exemplary drawing created by a test subject on a digital device for assessment of Alzheimer's disease in the elderly population, according to an embodiment of the subject invention.

FIG. 15 is an exemplary diagram showing locomotion of a test subject in a gait measurement test, according to an embodiment of the subject invention.

### DETAILED DISCLOSURE OF THE INVENTION

According to the embodiments of the subject invention, a machine-learning (ML) based motion analysis and training method and systems are provided.

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention pertains. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

When the term “about” is used herein, in conjunction with a numerical value, it is understood that the value can be in a range of 90% of the value to 110% of the value, i.e. the value can be +/−10% of the stated value. For example, “about 1 kg” means from 0.90 kg to 1.1 kg.

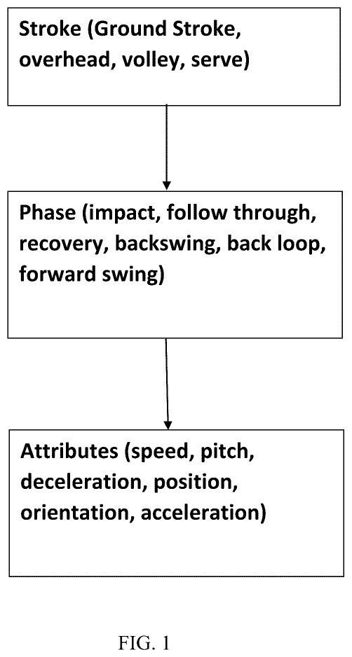

Referring to FIG. 1, movement measurement data is collected during training, matches or regular performance. The data is processed to extract movement patterns relevant to the tennis strokes. The motion data is parsed to extract the segments/phases associated with the primary actions and information associated with their outcomes. The patterns are classified to extract different categories and classes of motion patterns as shown in FIG. 1.

The diagrams in FIG. 1 show different categories, classes and types of tennis strokes. For example, the strokes can be classified into categories including groundstroke, overhead, volley, serve or other types including forehand strokes or backhand strokes.

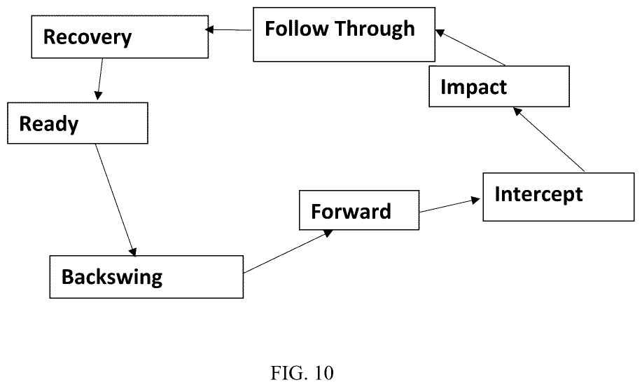

The tennis player's movements can be modeled as a sequence of dynamics with initial and goal sets. Therefore, one of the goals is to segment the larger movement units (for example, the entire tennis stroke) into the subunits or phases as shown in FIG. 10. For instance, a stroke such as a forehand groundstroke can be segmented into different phases including impact, follow-through, recovery, backswing, back-loop, and forward swing, which can be characterized by different attributes such as speed, pitch, acceleration/deceleration, position, orientation, or rotation as shown in FIGS. 1 and 10.

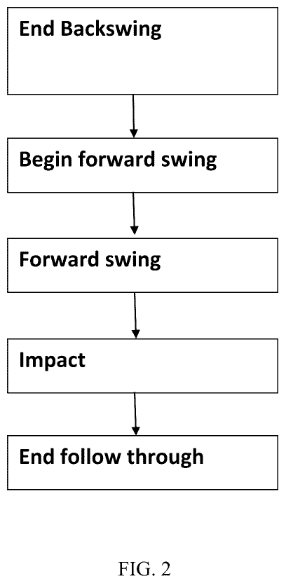

As shown by FIG. 2, the ML-base tennis skill analysis method and system according to embodiments of the subject invention provide three main functionalities.

### 1. Self-Evaluation

First, the skill levels of the tennis player can be evaluated and automatically classified into different levels, such as beginner, intermediate, or advanced level, by the ML-base tennis skill analysis method and system based on differences of the motions of players of different skill levels. Subsequently, an instruction module of the method and system can provide the user real-time feedback containing skill evaluation/classification information. Then, based on the user's historical data, the instruction module informs him/her whether he/she has made any progresses over time or whether he/she has reached a desired skill level.

### 2. Self-Analysis

Second, the larger movement units (for example, the entire tennis stroke) of the user can be automatically segmented by the ML-base tennis skill analysis method and system into subunits or phases as shown in FIG. 10. For instance, a stroke such as a forehand groundstroke can be segmented into different phases including impact, follow-through, recovery, backswing, back-loop, and forward swing. All the phases can be characterized by different attributes such as speed, pitch, acceleration/deceleration, position, orientation, or rotation. As a result, the instructional module of the method and systems can offer users real-time analysis, including a determination of whether their movements conform to the standard movement features of tennis techniques.

### 3. Self-Improvement

Third, motion similarity and motion difference of different skill level players are determined by the instruction module of the ML-base tennis skill analysis method and system of the subject invention. As a result, the instruction module can provide the user information of specific movement features that are crucial to improve performance or outcome and can also be used to protect the user from injury based on analysis of the motion data, allowing enforcement of features that can maximize the outcome of the user's tennis racket movements.

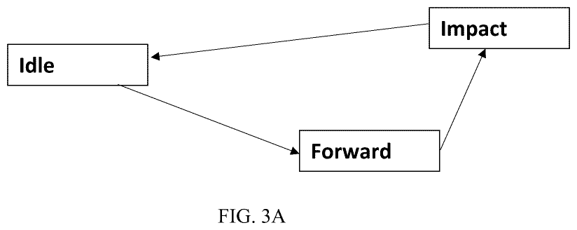

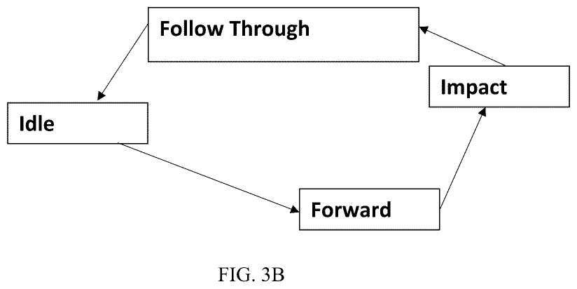

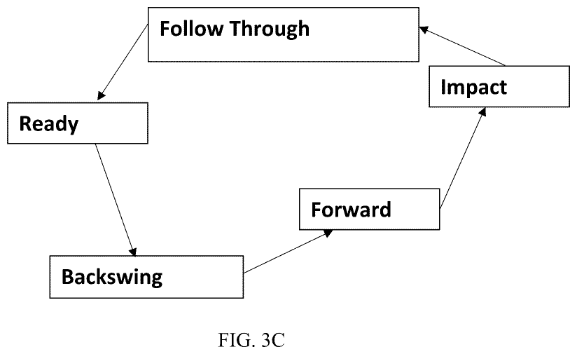

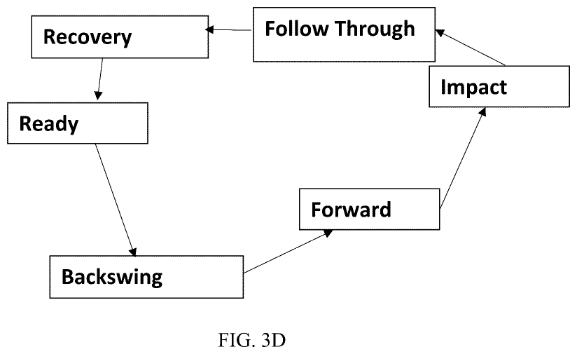

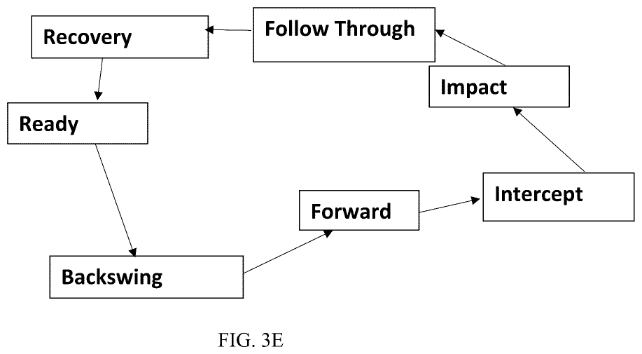

FIGS. 3A-3E illustrate the development of movement pattern structure with respect to phases of the tennis strokes. The model structure is expected to change over various stages of skill acquisition as shown in FIGS. 3A-3E.

For example, for a tennis player of the beginner level, stroke movements are mostly undifferentiated forward swing motions with the primary goal of intercepting the oncoming ball. Thus, a stroke of a beginner tennis player generally comprises three phases including idle, forward swing, and impact (the tennis racket hitting the tennis ball) with primary focuses on the forward swing and the impact as shown in FIG. 3A. Accordingly, more delicate phases of movements, such as the backswing which is essentially a power-loading movement, and the follow-through which is a consequence of the impact, are not integrated into the movements of the stroke.

On the other hand, a tennis player of intermediate level knows how to differentiate their motions for different effects on the ball and adjust to the impact. In reality, the tennis player discovers these new stroke patterns either by himself/herself through adaptation of his/her technique or through the direction of a tennis coach. For more specific outcomes and demanding conditions, the stroke must be optimized. For example, to achieve strong topspin, in the phases preceding the impact (backswing and forward swing), the stroke trajectory must follow a pattern that leads to impact conditions generating topspin. For example, the racket head must drop (“back-loop”) low enough before the forward-swing initiation. As a result of this movement refinement process, the overall movement pattern evolves with skill. Therefore, a stroke of an intermediate tennis player generally comprises the follow-through and backswing as part of a coherent stroke motion as shown in FIG. 3C or the recovery and the back loop as shown in FIG. 3D. Phases of the stroke can be expanded to include the follow-through and/or backswing, stroke initiation and interception, recovery phases, backswing, and back-loop.

Thus, one of the goals of the ML-base tennis skill analysis method and system of the subject invention is to be able to identify different phases of the stroke model from the data in order to account for tennis skill levels of the user.

The new primary phases are shown in FIGS. 3A-3E in dashed lines, and the new expanded phases are highlighted by a dashed line surrounding these phases. The distinction between the addition or expansion phases is determined by the different movement patterns.

The movement segmentation for the evolving movement patterns shown in FIGS. 3A-3E can be extracted from individual users by ML-models in a data-driven fashion. These models can be stored in a data library to enable tracking of the skill development process. It is desirable to model long-term skill acquisition with data from a sufficiently broad skill population (in terms of style and technique) and knowledge about the skill acquisition process (in terms of pattern formation). The understanding of the development of the movement patterns can be applied to refining an individual's motion structure.

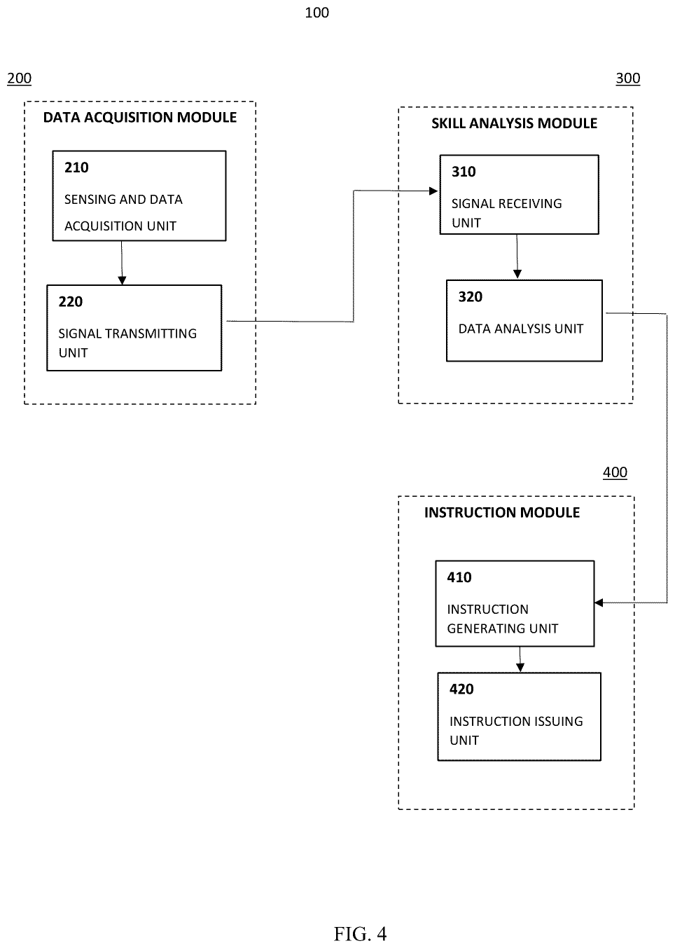

FIG. 4 illustrates a ML-base tennis skill analysis and training method and system 100 according to embodiments of the subject invention. The ML-base tennis skill analysis method and system 100 includes a data acquisition module 200 for measuring kinetic or kinematics of a test subject; a skill analysis module 300 for wirelessly communicating with the data acquisition module 200 and analyzing the measurements to evaluate patterns of hand/arm movements of the test subject; and an instruction module 400 for providing feedback and/or advice for improvement to the user with respect to skills of the hand/arm motions.

The data acquisition module 200 as shown in FIG. 4 includes a sensing and data acquisition unit 210 and a signal transmitting unit 220. The sensing and data acquisition unit 210 measures and records kinetic and/or temporal features of the movements through inertial measurements.

In an embodiment, the data acquisition module 200 can be an Apple Watch™ having an inertial measurement unit (IMU) put on a wrist or other positions of the test subject's forearm, measuring and reporting specific force/acceleration, the change in velocity over time, and angular motion rate, when the test subject swings a tennis racket.

It is noted that previous studies have demonstrated that human swing examined using ground reaction forces has slow dynamics and a sampling frequency greater than 10 Hz is sufficient for measuring kinetic or temporal parameters of tennis swing movements of a human being test subject. Thus, the data acquisition module 200 can operate at a sampling frequency of, for example, 30 Hz, though embodiments are not limited thereto.

In one embodiment, to ensure accurate data sensing and recording, the sensing and data acquisition unit 210 is calibrated before the measurements by applying known loads to the transducer and recording the output values to determine calibration relationships.

When the measurements are acquired by the sensing and data acquisition unit 210, the signal transmitting unit 220 transfers wirelessly or by wire the measurment results to the skill analysis module 300 such that the measurement results are analyzed by the skill analysis module 300 for determining the motion patterns of the test subject.

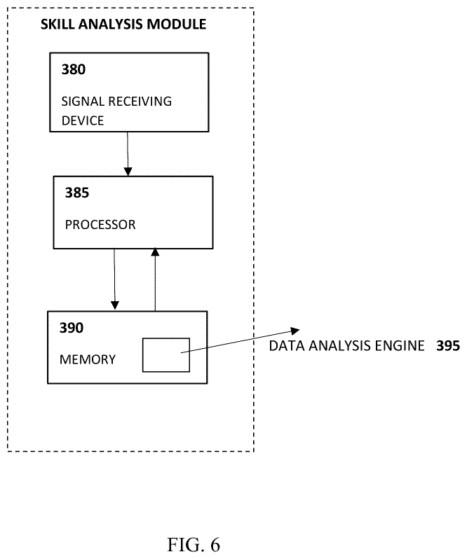

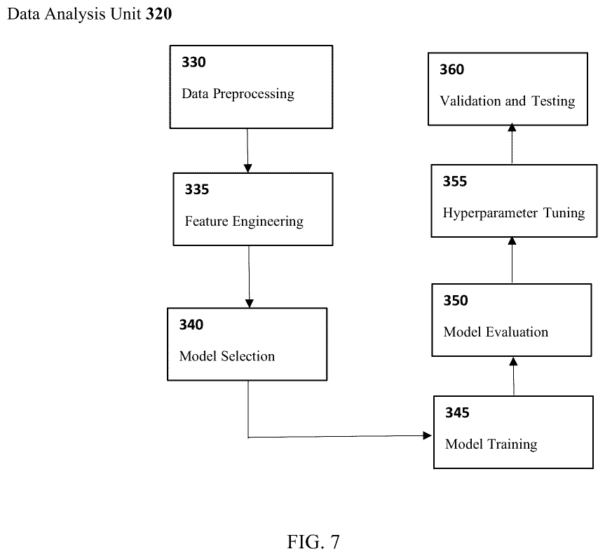

The skill analysis module 300 includes a signal receiving unit 310 and a data analysis unit 320. The signal receiving unit 310 receives data or signals transmitted from the signal transmitting unit 220 and the data analysis unit 320 performs data analysis steps as described in further detail below with reference to FIGS. 6 and 7.

Moreover, the instruction module 400 comprises an instruction generating unit 410 and an instruction issuing unit 420. The instruction generating unit 410 is configured to generate instructions/feedback to the user based on the results of the data analysis unit 320 for the test subject to achieve desirable tennis skills. The instruction issuing unit 420 is configured to display the instructions/feedback to the test subject in forms of text, audio, image, or video.

According to one embodiment, the sensing and data acquisition unit 210 comprises the Inertial Measurement Unit (IMU) which is a device comprising accelerometers to measure and report specific force/acceleration, or the change in velocity over time, and gyroscopes to measure and report angular motion rate.

A wearable device equipped with IMU such as an Apple Watch™ having an accelerometer of up to 32 g-forces with fall detection and a gyroscope is employed for tests during which the wearable device makes constant contact with the user's body part such as a wrist that generates motions for testing purposes.

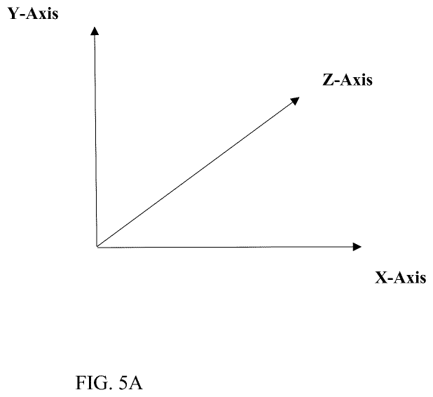

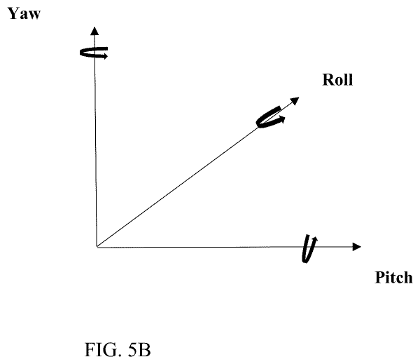

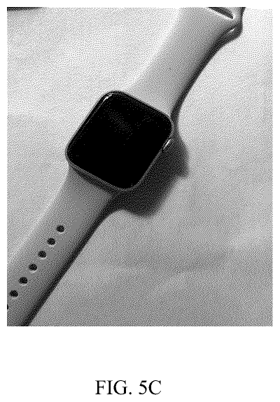

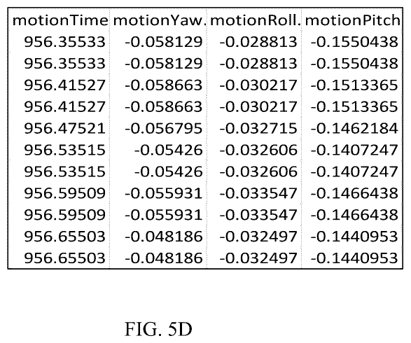

In particular, the accelerometer and the gyroscope of the wearable device facilitate determination of orientation. For instance, there are three separate accelerometers in the wearable device—one for each axis, specifically the x-, y-, and z-axis as shown in FIG. 5A. In addition, the three accelerometers detect movements in any direction and the gyroscope measures the rate of rotation (“angular rate”) across these three axes as shown in FIG. 5B.

Thus, the IMU comprising a 3-axis accelerometer and a 3-axis gyroscope is a 6-axis system as it provides two different measurements along each of the three axes for a total of six measurements.

The accelerometer sensor measuring the acceleration (differential rate of variations of velocity) applied to the wearable device in meter per square second (m/s²) in the local Cartesian device coordinates.

When the IMU device rests in a place on a table in parallel to the earth surface, the accelerometer of the device read a magnitude of 9.81 m/s², the gravity of the earth. On the other hand, when the IMU device is in free-fall and therefore accelerating towards the ground at gravity, its accelerometer reads a magnitude of 0 m/s².

In order to measure the real acceleration of the device, also referred to as linear-acceleration, the contribution of the force of gravity must be eliminated. Applying a high-pass filter to the results of accelerometer readings eliminates the slow changes of the readings caused by gravity and detects the fast changes introduced by movements of the device. Conversely, using a low-pass filter isolates the force of gravity and eliminates the fast changes in the acceleration caused by the movements of the device.

Gyroscope sensor measures the differential rotation momentum around the local x, y, z axis of the device Cartesian coordinates in radians/second (rad/s). The output of the gyroscope is integrated over time to calculate a rotation describing the change of angles over time. Rotation is positive in the counterclockwise direction. As a result, an observer looking from some positive location on the x, y or z axis at a device positioned on the origin reports positive rotation if the device rotates counterclockwise.

According to previous investigation [1], studies were carried out to evaluate the pre-processed (raw) data from an Apple Watch™ for validation of its inertial and heart rate sensors. Results were compared with an in-house 9DOF (NineDegrees of Freedom) IMU and Polar H10™. All correlations were greater than 0.95 and all data sets showed high level of agreement using Bland-Altman analysis. Therefore, the measurements of the acceleration sensor and the Gyroscope of the Apple Watch™ were determined to be highly accurate.

In one embodiment of the subject invention, a third-party software APP named “SensorLog” (sensorlog.berndthomas.net) is used for data sampling and recording. SensorLog APP records the raw sensor data and generated csv (comma-separated values) files. The recorded data from the wearable device such as an Apple Watch™ is then transmitted, for example, using Bluetooth®, to the skill analysis module 300 which may be a smart phone (for example, iPhone™) or other suitable computing means for data analysis.

The field tests were conducted by test participants ranging in age from 8 to 18 years old. Participants were informed of the reasons for the study and signed a consent form to participate in the study. The field tests were conducted at surfaced tennis courts under clear weather conditions.

Although the maximum sampling rate in the Apple Watch™ is 500 Hz, the actual sampling rate of the data collection is 33 Hz due to the limitation of the SensorLog APP utilized for the tests. Participants were asked to stand motionless for 5 seconds at the beginning of the tests, then practice a predetermined number of tennis strokes (for example, 10 forchand strokes or 20 forehand strokes) and then stop and stand motionless for 5 seconds.

FIG. 5C is an image of an Apple Watch™ used as an embodiment of the data acquisition module 200. The Apple Watch™ depicted in FIG. 5C is built based on a Texas Instruments Internet-of-Things prototyping platform having a 12-Bit ADC and 256 kB of RAM. On-board data recording is built-in with a micro-SD flash card capable of memory expansion up to 512 GB. This ancillary data acquisition route takes precedence when wireless data streaming is either not guaranteed or not needed. A CC3200 low-power Wi-Fi module is utilized for wireless communication.

In the field tests, 11 human being test subjects, between 8 and 18 years of age and of all genders/races, were asked to play tennis strokes while wearing an Apple Watch™ on the wrist of their hands holding the tennis racket for motion sensing and data acquisition. This setup allows for motion sensing and data acquisition.

Half of the test subjects are beginner level tennis players and the other half are intermediate level players.

Because minors are the vulnerable population, the parents of minor test subjects are asked to sign a physical paper consent form for the minor test subjects to participate. The test subjects are voluntary to drop out at any time during the project. The consent form describes the test purpose, risk and benefits of the tests, allowing voluntary participation and withdrawal at any time from the study, an option to participate in the test, and a signature indicating the parent's permission of their minor children's participation.

Names of the test subjects are collected for identification. However, the data are confidential and each name is identified with a numerical ID for the data analysis purpose. The data are stored on a password protected private storage device on a personal computer for data collection and analysis. Only the co-inventors are allowed access to the data.

The participating minor test subjects are first asked to complete a questionnaire to assess related background tennis information about themselves, such as their universal tennis rating (UTR) or if they had a private coach before. Then, they are invited to practice a set number of tennis swings for a maximum of 20 minutes with at least three breaks, while wearing an Apple Watch™ on their wrist. The Apple Watch™ is equipped with sensors needed for this project to record the movement data of the test subjects. There are no major potential risks associated with this project, other than the common risks of tennis playing, such as slipping on the court. The risks are minimized by providing protective gear such as wristbands and knee pads to the test subjects, providing written reminders to the test subjects informing the risks of the project and asking the test subjects to wear proper sports gear (for example, wearing tennis shoes instead of sandals).

In the field tests, each test subject is instructed to hit a specific number of balls wearing the Apple Watch™ for a maximum of 20 minutes. The tennis balls are gently fed for the test subjects to hit. The Apple Watch™ senses and collects the data. When the tests are done for the test subject, all the collected data are transferred to a computing device such as a smart phone or a laptop computer for analysis.

FIG. 5D shows data of a group of 14 motion features extracted from 82 features recorded by the SensorLog APP. For example, the extracted features include acceleration rates of the wearable device in contrast to acceleration by gravity along 3 axes of the reference frame and rotation rates of the wearable device, as shown in Table 1 below.

TABLE 1
Extracted Motion Features

| | Feature Name/Unit |
|---|---|
| 1 | motionTimestamp_sinceReboot(s) |
| 2 | motionRotationRateX(rad/s) |
| 3 | motionRotationRateY(rad/s) |
| 4 | motionRotationRateZ(rad/s) |
| 5 | motionYaw(rad) |
| 6 | motionRoll(rad) |
| 7 | motionPitch(rad) |
| 8 | motionUserAccelerationX(G) |
| 9 | motionUserAccelerationY(G) |
| 10 | motionUserAccelerationZ(G) |
| 11 | motionQuaternionX(R) |
| 12 | motionQuaternionY(R) |
| 13 | motionQuaternionZ(R) |
| 14 | motionQuaternionW(R) |

Note:
Quaternions are an alternate way to describe orientation or rotations in 3D space using an ordered set of four numbers. They have the ability to uniquely describe any three-dimensional rotation about an arbitrary axis and do not suffer from gimbal lock.

In alternative embodiments, the skill analysis module 300 can perform its functionalities based on another implementation as illustrated by FIG. 6. In contrast to FIG. 4, the data analyzer 300 shown in FIG. 6 includes a processor 385 and a memory 390 instead of the data analysis unit 320. The memory 390 can comprise executable-code components of a data analysis engine 395 for execution by the processor 385. The data analysis engine 395 performs the data analysis steps of as described in further detail below with reference to FIGS. 7, 8, 11, and 12. In this respect, the terms “executable” or “for execution” refer to forms of executable-code that can ultimately be run or executed by the processor 385, whether in source, object, machine, or other form.

As discussed above, a data analysis engine 395 may be embodied, at least in part, by software or executable-code components for execution by general purpose hardware. Alternatively, the same can be embodied in dedicated hardware or a combination of software, general, specific, and/or dedicated purpose hardware. If embodied in such hardware, each can be implemented as a circuit or state machine, for example, that employs any one of or a combination of a number of technologies. These technologies can include, but are not limited to, discrete logic circuits having logic gates for implementing various logic functions upon an application of one or more data signals, application specific integrated circuits (ASICs) having appropriate logic gates, field-programmable gate arrays (FPGAs), or other suitable components.

Referring to FIG. 7, the data analysis module 320 comprises a machine-learning pipeline which is a systematic sequence of data processing and modeling steps that are organized in a workflow to automate and streamline the process of developing, training, evaluating, and deploying machine learning models.

In one embodiment, the machine-learning pipeline comprises a step of data preprocessing 330 configured to prepare the raw data for analysis; a step of feature engineering 335 configured to create relevant features from the raw data that can be used to train the machine learning model;

a step of model selection 340 configured to select an appropriate machine learning algorithm/method or suitable model architecture; a step of model training 345 configured to train the selected model based on the preprocessed data; a step of model evaluation 350 configured to assess the performance of the trained model based on appropriate evaluation metrics; a step of hyperparameter tuning 355 configured to fine-tune the hyperparameters of the model/method to improve its performance; and a step of validation and testing 360.

In particular, the step of data preprocessing 330 may include one or some or all of the steps including data cleaning, handling missing values, data transformation, and/or feature extraction. The step of feature engineering 335 may include one or some or all of the steps including dimensionality reduction, scaling, encoding categorical variables, and/or creating new features. The step of model training 340 comprises feeding the data into the selected model and adjusting the model parameters to learn patterns from the data. In the step of model evaluation 345, the evaluation metrics may include accuracy, precision, recall, F1-score, area under the ROC curve (AUC-ROC), or other suitable metrics. The hyperparameters of the step of hyperparameter tuning 355 are settings that are not learned during the model training, such as learning rate, regularization strength, and/or the number of hidden layers in a neural network. In the step of validation and testing 360, the data are split into a training set, a validation set, and a testing set to evaluate the model's performance on unseen data. The validation step facilitates selecting the best model among a plurality of candidate models, while the testing step provides a final estimate of the generalization ability of the model selected.

The machine learning pipeline is designed to optimize the productivity, reproducibility, and scalability in the development of machine learning models, enabling efficiently iteration on different approaches, experimenting with various features and models, and ultimately making reliable and accurate predictions for real-world applications in the tennis skill analysis.

### Data Preprocessing

Data preprocessing is the process of cleaning, transforming, and organizing raw data into a format that is suitable for analysis or for training machine learning models. Data preprocessing is essential because raw data often contains noise, inconsistencies, missing values, and other issues that can hinder the accuracy and effectiveness of data analysis or machine learning algorithms.

The data preprocessing may involve data cleaning such as identifying and handling missing values, outliers, and erroneous data points; and/or data transformation through which raw data are transformed to make it more suitable for analysis or modeling (for example, features are scaled to have a consistent range by standardization or normalization and encoding categorical variables into numerical values).

### Feature Selection/Feature Engineering

Feature selection/feature engineering is the process of removing irrelevant input features when developing the ML-based model to both reduce the computational cost of modeling and to improve the performance of the model.

### Model Selection

Commonly used classification methods include:
1. Support vector machine (SVM),
2. Logistic regression,
3. Naïve Bayes,
4. K-Nearest neighbors,
5. Decision tree.

Support Vector Machine (SVM) is adopted for classification by finding a hyperplane that best separates data points belonging to different classes while maximizing the margin between the classes. SVMs is effective in High-Dimensional Spaces to perform well in high-dimensional spaces (a large number of features). Since SVMs aim to maximize the margin between classes, leading to better generalization on unseen data and less prone to overfitting compared to some other classification methods.

Kernel Trick: SVMs can handle non-linearly separable data by using kernel functions that map the original data into a higher-dimensional space. This allows SVMs to capture complex relationships between features.

Moreover, SVMs model is robust to outliers and are less affected by outliers in the training data because they focus on the support vectors (data points that are closest to the decision boundary) when determining the optimal hyperplane.

Further, SVMs aim to find the global optimum of the margin, which is often desirable in practice, as it reduces the risk of getting stuck in local optima during training. The potential limitations of the SVM model include:
1. Sensitivity to Scaling for data preprocessing: SVMs are sensitive to the scale of input features. It's important to standardize or normalize the data before applying SVMs to ensure that all features contribute equally to the decision boundary.
2. Computational Complexity: Training SVMs can be computationally expensive, especially when dealing with large datasets or high-dimensional feature spaces. The time complexity is typically between O(n\hat{ }2) and O(n\hat{ }3), where “n” is the number of data points.
3. Memory Intensive: SVMs require storing the support vectors in memory, which can be memory-intensive when dealing with a large number of support vectors.
4. Parameter Tuning (to move to tuning): SVMs have several hyperparameters that need to be tuned, such as the choice of kernel, regularization parameter (C), and kernel-specific parameters. Finding the right combination of hyperparameters can be challenging and requires careful experimentation.
5. Interpretability: The decision boundary generated by SVMs can be complex, especially when using non-linear kernels, which can make it challenging to interpret the model's decisions.

### Model Training

The goal of model training is to find the optimal hyperplane (decision boundary) that maximizes the margin between the classes while minimizing classification errors. The optimization process involves solving a quadratic programming problem to determine the support vectors and their associated weights. The margin is defined as the distance between the hyperplane and the nearest data points from each class. The SVM aims to maximize this margin. During the model training, regularization parameter (C) can be adjusted to control the trade-off between maximizing the margin and minimizing classification errors. A smaller C encourages a wider margin but allows some misclassification, while a larger C penalizes misclassification more heavily and results in a narrower margin.

### Model Evaluation

The testing/validation sets are utilized to assess the model's performance. Common evaluation metrics for classification problems include accuracy, precision, recall, F1-score, and ROC curves.

For classification tasks, the ROC (Receiver Operating Characteristics) curve, and specifically the Area Under the Curve (AUC) for the ROC curve, stand out as essential metrics for assessing the performance of a classification model.

AUC-ROC curve is a performance measurement for the classification problems at various threshold settings. ROC is a probability curve and AUC represents the degree or measure of separability. It tells how much the model is capable of distinguishing between classes. Higher the AUC, the better the model is at predicting 0 classes as 0 and 1 classes as 1. In simpler terms, the higher the AUC, the better the model is at distinguishing between tennis players who possess specific motion skills and those who do not possess these skills.

The ROC curve is constructed by plotting True Positive Rate (TPR) on the y-axis against False Positive Rate (FPR) on the x-axis, providing a visual and quantitative insight into the model's pattern discrimination capabilities.

### Hyperparameter Tuning

SVMs have several hyperparameters that need to be tuned, such as the choice of kernel, regularization parameter (C), and kernel-specific parameters. Experiments are conducted to find the right combination of hyperparameters to improve performance and avoid the overfitting issue.

### Validation and Testing

To ensure a certain level of confidence that the selected model can accurately discern meaningful patterns within the data while minimizing the effects of noise, a model with low bias and low variance is desired.

To achieve this goal, the training data is partitioned and a portion of the training data is reserved as validation data for obtaining model predictions, distinct from the data used for model training. This error estimation provides insight into how well the model performs on unseen data or a validation set, employing a straightforward form of cross-validation technique.

However, there is a constant challenge when it comes to setting aside a portion of the data for validation, as there are only a relatively small number of data collected for model training. Reducing the training data runs the risk of missing essential patterns or trends within the dataset, subsequently increasing error due to bias. Therefore, K-Fold cross-validation methodology is employed which ensures sufficient data for model training while also reserving enough data for model validation.

In K-Fold cross-validation, the data is divided into k subsets. The holdout method is then repeated k times, with each iteration using one of the k subsets as the test or validation set, while the remaining k-1 subsets are combined to form the training set. The error estimation is averaged across all k trials to provide a comprehensive assessment of our model's effectiveness. Notably, K-Fold cross-validation guarantees that every data point participates in the validation set exactly once and in the training set k-1 times. This approach substantially mitigates bias, as most of the data is used for model fitting, and it also reduces variance, as a substantial portion of the data is employed for validation. Alternating the roles of training and test sets further enhances the effectiveness of this methodology. While a common guideline suggests using K=5 or 10, it is not a fixed value and can be adapted to the specific context.

Cross-validation is an excellent technique for evaluating the performance of the ML model, particularly when overfitting is a concern. Additionally, it aids in the fine-tuning of the ML model's hyperparameters by identifying parameter configurations that result in the lowest test error. This foundational knowledge facilitates using various validation techniques, all readily accessible through Scikit-Learn, which provides a straightforward Python code to facilitate the model development.

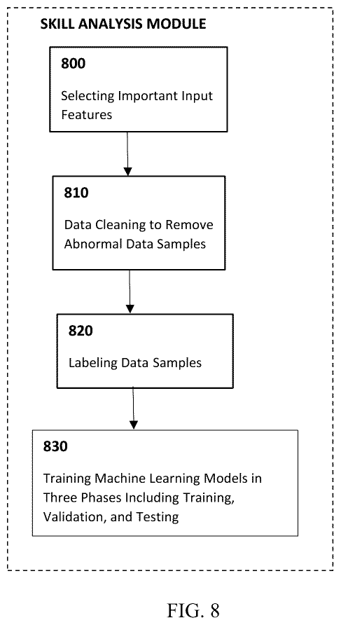

Referring to FIG. 8, for tennis skill level identification, the data are collected from participants. In step 800, important input features such as Yaw, Roll, Pitch are selected. Then, in step 810, data cleaning is performed to remove abnormal data samples. Next, in step 820, the data samples are labeled such that each frame of the data series is labeled corresponding to the participant's tennis skill level such as the beginner level or the intermediate level. Then, in step 830, the machine learning model, such as a support vector machines (SVM) model that fits an optimal hyperplane between datasets, is trained in three phases including training, validation, and testing.

The output of the machine learning model is the percentage based on the number of frames that is classified as the beginner level or the intermediate level. For example, for a data series having 1000 frames, if among the 1000 frames, 800 frames are classified as intermediate level, the percentage of intermediate level is 80% (800/1000) which is higher than 50%. Consequently, the level of the tennis player is classified as intermediate.

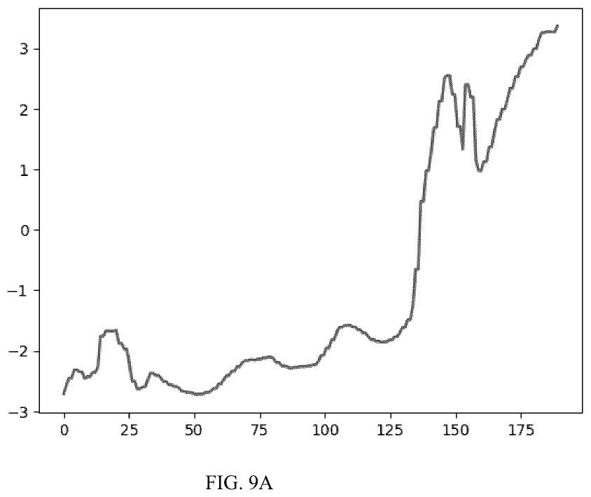

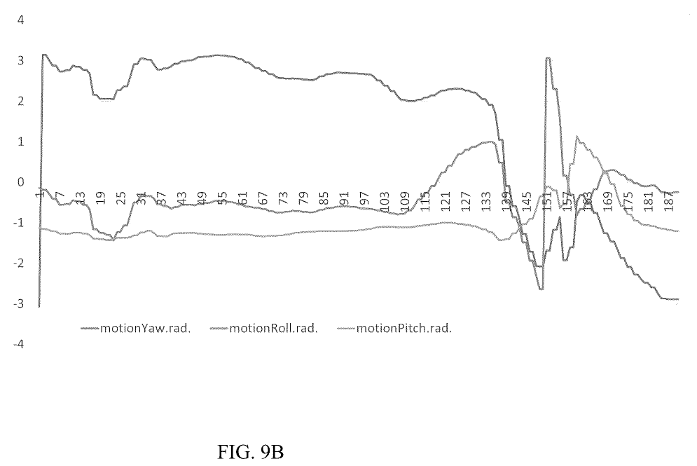

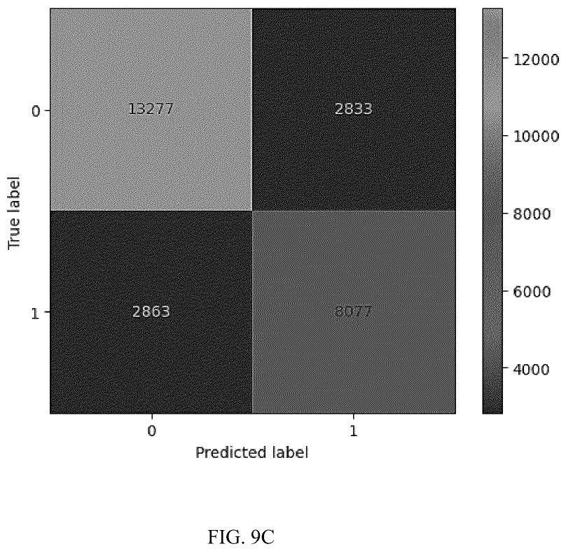

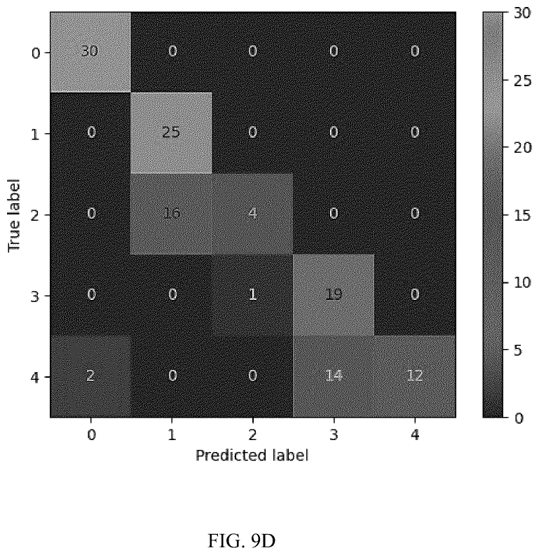

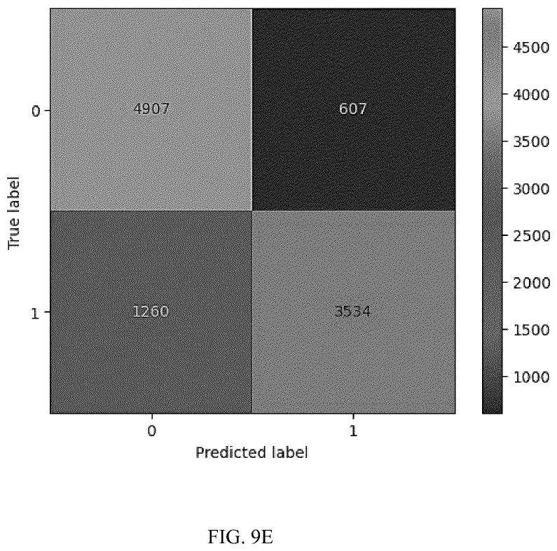

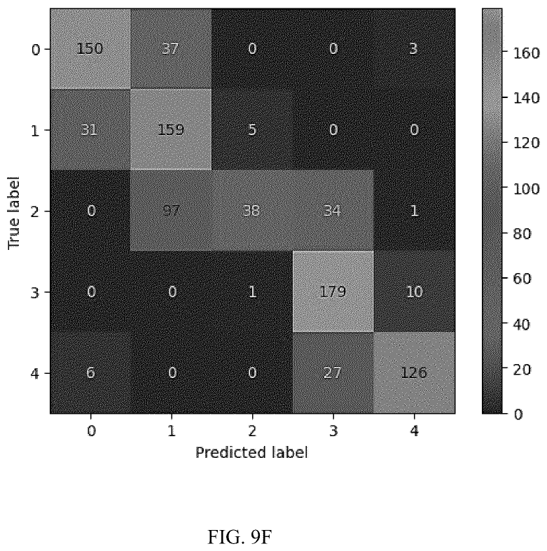

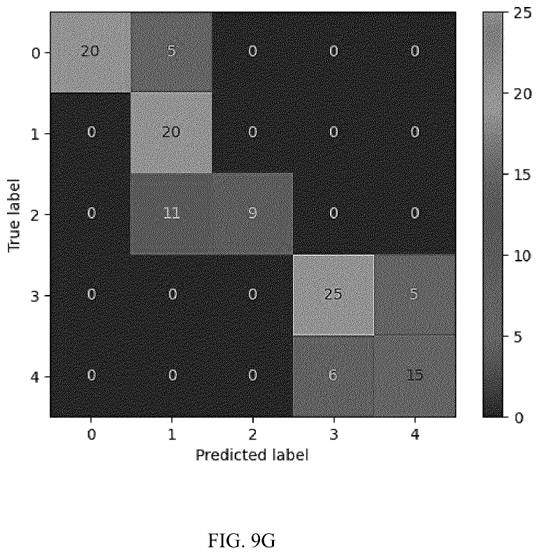

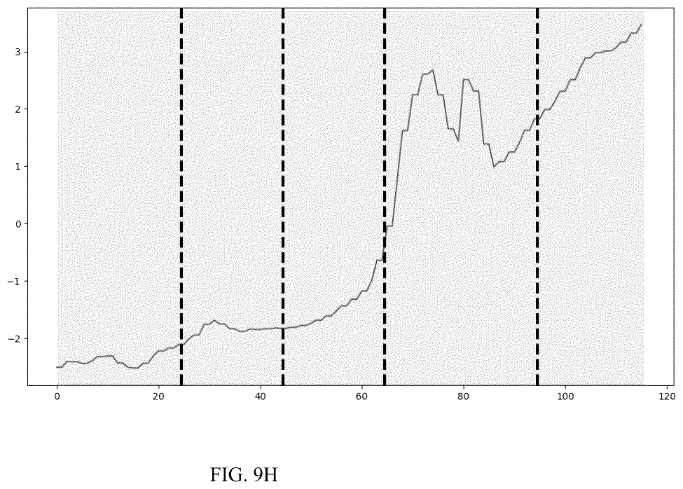

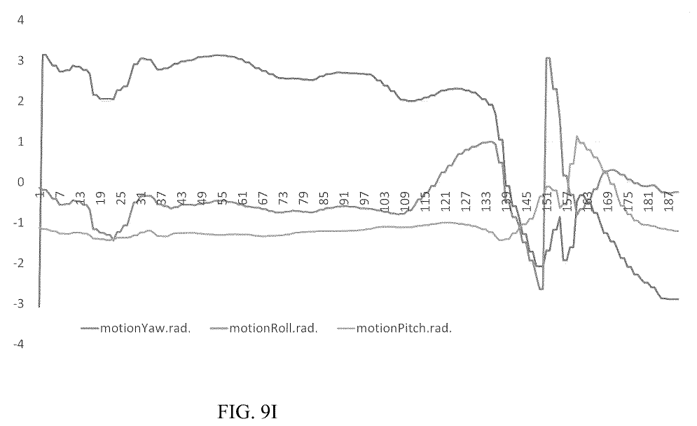

FIGS. 9A-9C show the data collected from a series of forehand ground strokes performed by a first tennis player who is manually labeled as a beginner level player. In particular, FIG. 9A illustrates the correlation between yaw motions in the strokes and time, FIG. 9B illustrates the correlation between roll motions in the strokes and time, while FIG. 9C illustrates the correlation between pitch motions in the strokes and time.

FIGS. 9D-9F show the data collected from a series of forehand ground strokes performed by a second tennis player who is manually labeled as an intermediate level player. In particular, FIG. 9D illustrates the correlation between yaw motions in the strokes and time, FIG. 9E illustrates the correlation between roll motions in the strokes and time, while FIG. 9F illustrates the correlation between pitch motions in the strokes and time.

The data presented in FIGS. 9A-9C and FIGS. 9D-9F serve as the input for training the SVM model.

FIGS. 9G-9I show the data collected from forehand ground strokes of a third tennis player whose level of skills is determined by the SVM model in the test phase as beginner level. In particular, FIG. 9G shows relationship between the yaw motions of the strokes and time. FIG. 9H shows relationship between the roll motions of the strokes and time. FIG. 9I shows relationship between the pitch motions of the strokes and time.

FIGS. 9G to 9I show data collected from a series of forehand ground strokes performed by a tennis player whose skill level has been classified as beginner according to the SVM model during the test phase. FIG. 9G illustrates the correlation between yaw motions in the strokes and time, FIG. 9H illustrates the correlation between roll motions in the strokes and time, while FIG. 9I illustrates the correlation between yaw motions in the strokes and time.

FIG. 10 illustrates the stroke model with the seven phases and examples of transition rules.

Each phase is characterized by particular kinematic and dynamic characteristics. These characteristics can be used to formulate detection rules, which can be applied to automatically segment the phases from the motion data. The detection rules also define the state transition in the finite-state model. Note that for many of these rules the measured quantities (here RBF angular rates and acceleration) will give adequate phase transition information only some of the time. Therefore, some phase transition rules also include estimated quantities such as the orientation.

The transition rules between the phases of the strokes are as follows:

Ready: the ready state or phase corresponds to the time interval between the end of the recovery to the beginning to the initiation of the next stroke, that is, the beginning of the backswing until the next stroke. The racket motion during the ready phase is quasi stationary, therefore the ready phase period can be determined from the magnitude of the acceleration and angular rate measurements by a threshold on the total acceleration and angular rate.

Backswing: the backswing corresponds to the first phase of a new stroke. As the name implies the state is initiated by the racket backswing motion. The initiation of the backswing phase can be detected by using the time the azimuth rate reaches a given threshold and azimuth has reached a threshold.

Back loop: the back loop is used to designate the transition phase between the backswing and the forward swing. This transition represents primarily a rotational motion from the backward swing motion to the forward swing motion. This phase can be extracted from the time interval involving relatively low acceleration but high angular rate, for example by evaluating the (quaternion unit sphere) trajectory curvature.

Forward swing: the forward swing is the phase that leads to the impact. Therefore, it requires sufficient racket head speed and precise contact conditions. It starts with the angular acceleration of the racket and ends with the ball interception and eventually impact. The start of the forward swing is the time when the high-curvature trajectory of the back loop changes into a low-curvature trajectory as the racquet is set on a ballistic path.

Impact: the impact onset is characterized by an impulsive acceleration associated with the momentum transfer that occurs when the ball strikes the string bed. As already described, the ball contact during impact lasts about 5 msec. The start of the impact is detected when the total acceleration ascends through the threshold.

The impact event is assumed to only last the period of time where the ball and racquet are in contact. Although the impact can be shown to be a nonlinear event (and therefore not guaranteed to be invariant in duration relative to impact strength), empirical study suggests that most impact events have a similar duration. Using duration is also preferable to examining rates/accelerations as it is not affected by racquet vibration modes.

Follow through: the follow through primarily involves keeping control of the racket immediately following the impact and slowing the racket. This motion is mostly characterized by the limits on biomechanical range of motion. Similar to the forward swing, the follow through is primarily characterized by the change in racket angular deceleration. The end of the follow through can be detected by the racket swing rate r reversing sign.

Recovery: during the recovery the player takes the racket from the end of the follow through to a resting state which designates the ready position.

These features and associated structure provide the basis for the skill analysis as well as for the design of player feedback mechanisms. The phases of a forchand tennis ground stroke include ready, backswing, back loop, forward swing, impact, follow through, and recovery. Respectively, they are abbreviated as: re, bs, bl, fs, imp, ft, and rec.

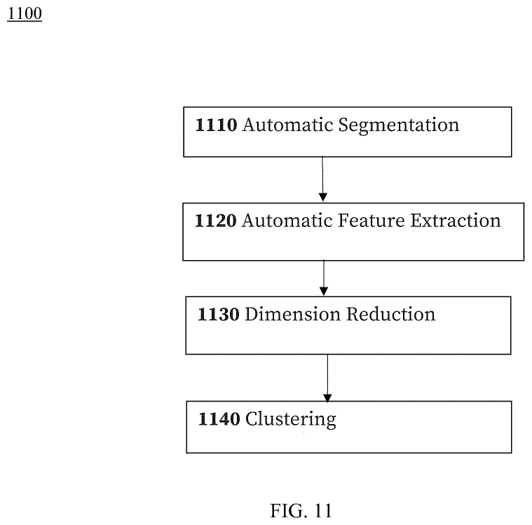

Referring to FIG. 11, the stroke phase identification module 1100 comprises four steps including, but not limited to, a step 1110 of automatic segmentation, a step 1120 of automatic feature extraction, a step 1130 of dimension reduction, and a step 1140 clustering.

### Automatic Segmentation

First, in the step 1110 of automatic segmentation, the available timeseries data are split into segments by an automatic segmentation method such as the pruned exact linear time (PELT) method or the Window (window-based change point detection) method which can be found in the Ruptures library that can be configured to detect changepoints (signal shifts) on each of the signals. For example, the data can be split according to signal shifts in the x-axis of acceleration rate based on a first optimal threshold. The optimal threshold values are determined through a process of experimentation and trial and error.

Change point detection is the process of identifying points in a time series where the underlying statistical properties of the data change abruptly. The changes can represent important events or transitions in the data, providing valuable insights for machine-learning based decision-making and prediction.

### Automatic Feature Extraction

Then, in the step 1120 of automatic feature extraction, an automatic feature extraction method such as Tsfresh is utilized to extract features or generate new features such as mean, variance, Fourier coefficients from the timeseries data collected. The automatic feature extraction method is applied to each of the segments to extract features for each segment. During the process, the raw data collected are transformed into useful features that better characterize the data, enabling the machine learning model to learn better from those features.

The features extracted capture different aspects of the data's statistical and temporal characteristics including, but not limited to, mean, standard deviation, skewness, kurtosis, various percentiles, and more.

Once the more meaningful features are extracted, any suitable feature selection method such as Tsfresh can be applied to reduce the feature set and only keep the most important features for machine learning. Not all extracted/generated features are relevant for the particular prediction task of the subject invention. A method such as Tsfresh includes automatic feature selection to identify the most informative features for the specific modeling needs of the subject invention, reducing dimensionality and potentially improving model performance. While Tsfresh provides default settings for feature extraction, the extraction process can be customized by specifying parameter values and criteria for feature selection.

### Dimension Reduction

Next, in the step 1130 of dimension reduction, a dimension reduction method such as PCA is applied to the data to reduce the number of dimensions to the most efficient number. The dimensionality reduction method reduces the number of features or dimensions in a dataset while preserving the most important information to simplify the problem, improve model performance, and reduce overfitting.

In one embodiment, the dimensionality reduction method such as PCA identifies the most important features (principal components) in the data. PCA transforms the original features into a new set of orthogonal (uncorrelated) features called principal components, simplifying the relationships between features, making it easier for the machine learning models to capture the underlying patterns in the data.

### Clustering

Then, in the step 1140 of clustering, clusters are determined from the dimensional data by a clustering method such as Kmeans. The clustering method of machine learning is configured for grouping data points into clusters based on their similarity for exploring the structure of data by grouping similar data points together and understanding the inherent patterns and relationships within the dataset.

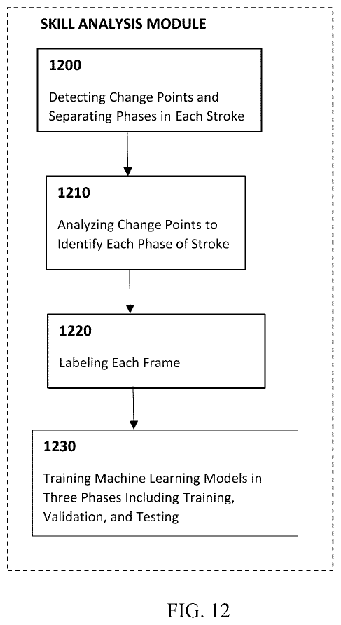

Now referring to FIG. 12, for tennis stroke phase segmentation, the data are collected from participants. In step 1200, change points are detected by machine learning techniques such as Ruptures library. Then, in step 1210, based on the results of the detection of change points, phases such as idle, impact, or forward-swing of each stroke are automatically segmented. Next, in step 1220, each frame is labeled to a corresponding phase. Then, in step 1230, the machine learning model is trained in three phases including training, validation, and testing.

The output of the machine learning model is the percentage based on the number of frames that is correctly labeled as a certain phase. For example, for a data series of a forward-swing phase having 500 frames, if among the 500 frames, 400 frames are classified as forward-swing phase, and the other 100 frames are classified as other phase(s). The correct percentage of automatic segmentation is 90% (400/500) which is higher than 50%. Consequently, player is identified as having correct tennis techniques. However, if the percentage is lower than 50%, the player needs to improve his/her tennis stroke techniques until the percentage is higher than 50%.

### Ruptures Library

Change point detection is the method of pinpointing instances in a time series where the fundamental statistical characteristics of the data undergo abrupt alterations. These transformations often signify critical events or transitions in the data.

The ruptures library is used for detecting changepoints (signal shifts) on each of the signals. Ruptures is a Python library for off-line change point detection, providing methods for the analysis and segmentation of non-stationary signals. The algorithms include exact and approximate detection for various parametric and non-parametric models. Moreover, thanks to its modular structure, different algorithms and models can be connected and extended within the Ruptures library.

Rupture detects regime changes within a signal by returning a list of breakpoints. Change point detection allows for recognition of significant shifts in the time series data, providing valuable insights for decision-making and prediction.

Ruptures library provides a diverse array of algorithms designed to uncover change points in time series data. Some of the widely used algorithms in the library include:

Binary Segmentation: This algorithm recursively splits the time series into two segments at the point that maximizes a chosen statistical criterion, such as the likelihood ratio or the sum of squared differences.

Pelt: The Pruned Exact Linear Time (PELT) algorithm efficiently pinpoints the optimal locations for change points while minimizing unnecessary splits.

Dynamic Programming: This approach offers an optimal method for identifying change points through dynamic programming techniques.

Multiple Change Point Detection: Ruptures identifies multiple change points within a time series, enabling the discovery of not just the initial change but all significant changes along the way.

Custom Cost Functions: Users have the flexibility to define their own cost functions to gauge the importance of a change at a specific point. This adaptability ensures that change point detection can be tailored to the unique characteristics of the data and its intended application.

Visualization: The library provides tools for visualizing the detected change points and segments, making it easier to understand and interpret the results.

Python Integration: Ruptures is implemented in Python, facilitating its seamless integration into the data analysis workflows, particularly if Python is already used for the data analysis and machine learning tasks.

Support for Different Data Types: Ruptures is not limited to time series data and can be used for change point detection in various types of data, including univariate and multivariate data.

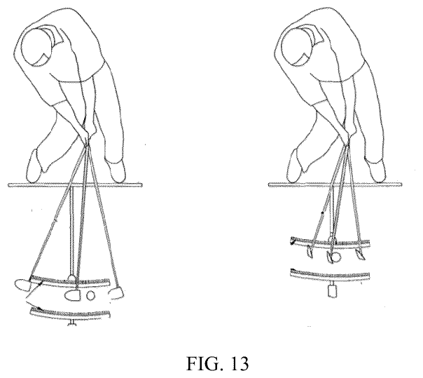

The ML-base tennis skill analysis method and system according to embodiments of the subject invention are configurable to generalize across many activities and train a wide range of movement types attributed to its ability to extract structure and track patterns from the performance histories of motion data. Exemplary applications may include various sports such as Ping Pong, badminton, golf (as shown in FIG. 13), pickleball, or other racket-based sports.

In other embodiments of the subject invention, the ML-base tennis skill analysis method and system can be applied to address issues of another domain—medical field. For instance, the approach can be employed to leverage the capabilities of machine learning in the realm of tennis skill analysis and be applied to the critical tasks of identifying cognitive impairments associated with conditions such as Alzheimer's disease, which stands as one of the most prevalent forms of dementia. The hallmark indications of Alzheimer's disease encompass a progressive deterioration in cognitive capacities along with compromised performance in activities of daily living.

One of the methods for assessment of Alzheimer's disease in the elderly population is digital drawing tests (DT) in which the participants are tasked with the replication of a presented figure. A body of clinical research has consistently demonstrated that suboptimal performance in this test can serve as an indicative metric for a variety of cognitive functions that encompass attention, spatial construction abilities, visual memory, executive functions, and the intricate interplay between eye-hand coordination. Given the fundamental correlation between cognitive decline and dementia, it logically follows that the various cognitive domains, including but not limited to visuospatial skills, sustained attention, and executive function, would likely exhibit impairment in tandem. Moreover, it is essential to underscore that even a seemingly straightforward task such as duplicating a figure demands the harmonious operation of the aforementioned cognitive domains to achieve successful completion. Specifically, the task mandates the engagement of the participant's attention on the reference figure, the mental visualization of its geometric attributes, and the subsequent translation of this mental representation into a physical drawing using their hand.

As dementia progressively undermines cognitive functions, the capacity to execute such tasks is significantly compromised, leading to observable disparities in task completion times between individuals with dementia and their cognitively healthy counterparts. Notably, individuals with dementia experience prolonged contemplation periods and manifest irregularities in their drawing patterns—manifestations that are inherently embedded within the drawing process and the resulting image itself.

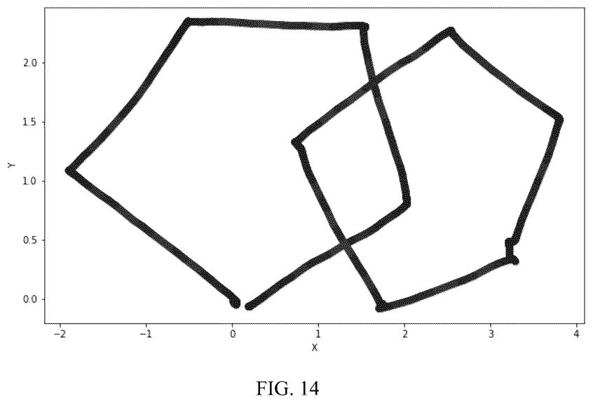

The Digital Drawing Test, with its diagnostic significance, has emerged as a valuable cognitive screening tool for a wide spectrum of dementia-related conditions, encompassing not only Alzheimer's disease but also other neurodegenerative disorders like Parkinson's disease. Referring to FIG. 14, the test paradigm involves a dual-pronged assessment wherein participants are prompted to draw a clock displaying a specific time (such as 10 minutes past 11, referred to as the “Command clock”) on a blank canvas. Following this, they are tasked with replicating a pre-drawn clock exhibiting the identical time (termed the “Copy clock”). The minutiae of the participant's wrist movements can be meticulously captured by a wearable smart device such as an Apple Watch™ worn on the wrist of the participant, enabling the capture of precise spatial and temporal data relevant to their drawing process.

By integrating the insights garnered from tennis skill analysis of subject invention with the intricacies of cognitive impairment evaluation, an encouraging avenue emerges to enhance the understanding of dementia and its carly identification. This innovative bridge holds promise not only for improving diagnostic accuracy but also for guiding therapeutic interventions and support strategies for individuals dealing with cognitive decline.

In certain embodiment of the subject invention, the ML-base tennis skill analysis method and system can be applied to assessing upper body motion asymmetry, serving as a valuable tool for aiding in the recovery process following physical injuries. This specific aspect can be employed for the evaluation of dissimilarities in upper body dynamics between opposing limbs, a condition often stemming from factors such as ailments, advancing age, clinical interventions, or inherent limb dominance.

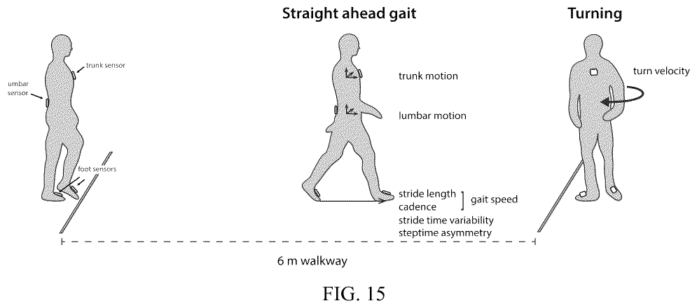

To illustrate, consider the application of this method to gauge upper body motion asymmetry in individuals undergoing recuperation from significant injuries such as cerebral palsy or stroke, as shown in FIG. 15. In these scenarios, adept therapists gather data within a controlled laboratory setting, utilizing a wearable smart device such as an Apple Watch™ worn on the wrist of the participant, to meticulously capture relevant motion-related information.

For instance, in a real-world rehabilitation scenario, a user who has endured a stroke and experiences paralysis on one side, may diligently endeavors to regain function in their right hand and arm. This endeavor involves putting an Apple Watch™ on the right wrist, collecting data from an array of sensors and relaying the data collected to a connected smart phone. Subsequently, the smart phone undertakes data processing, intricate calculations, and generates audio cues in response.

In one exemplary embodiment, while the course of rehabilitation encompasses an assortment of diverse movement sequences, focus may be centers on the intricate motions of the arm, encompassing reaching, grasping, and elevating the arm and hand.

The implementation of machine-learning-based motion analysis marks a significant advancement in the realm of sports technology, particularly in the context of tennis training. This cutting-edge approach involves the development of a high-performance prediction model tailored to enhance the overall training experience. Remarkably, in the current landscape, there is a notable absence of any commercial product utilizing artificial intelligence (AI) technology specifically designed for tennis training. This innovative application of AI not only underscores the pioneering nature of our solution but also addresses a crucial gap in the market, positioning our product as a trailblazer in revolutionizing how young tennis enthusiasts refine their skills and elevate their performance.

Another advantage is that the method and system of the subject invention enable the users to proficiently discern various phases within the stroke model through comprehensive data analysis. This pivotal capability allows the tennis players to tailor their assessments to the diverse skill levels and thus enhance their swing skills effectively. By accurately identifying and addressing specific phases of the stroke model, this innovative approach strives to provide personalized insights, thereby facilitating a more nuanced and targeted refinement of the user's tennis techniques.

Moreover, the subject invention provides a groundbreaking solution that enables seamless real-time data collection through the utilization of ubiquitous and cost-effective electronic devices, such as smartphones and Apple Watch™. This approach not only capitalizes on the widespread accessibility of smartphones but also harnesses their affordability, making advanced data collection capabilities available to a broader demographic. By leveraging these commonly owned devices, the invention ensures a practical and inclusive means of gathering information, revolutionizing the efficiency and accessibility of data-driven processes.

Therefore, the solutions according to the embodiments of the subject invention empower underprivileged children by providing them with a tool that enables independent cultivation and refinement of their tennis abilities. This initiative seeks to eliminate the significant financial barriers associated with hiring a personal coach or enrolling in a tennis clinic. By leveraging the widely available smartphones, skill development among young individuals who may not have had the means to pursue formal coaching is fostered. This inclusive approach not only unlocks the potential of aspiring tennis enthusiasts but also contributes to the broader mission of making sports education more equitable and accessible for all.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

[1] Espinosa H G, Thiel D V, Sorell M, and Rowlands D, Can We Trust Inertial and Heart Rate Sensor Data from an APPLE Watch Device? MDPI Proceedings, 2020, 49, 128
[2] Czaja, U.S. Patent No.: 11,612,787 B2.
[3] Bentley, U.S. Patent No.: 11,311,775 B2.
[4] May, U.S. Patent No.: 10,854,104 B2.
[5] Marquez, Patent No.: U.S. Pat. No. 10,555,689 B1.
[6] Tsoi, Patent No.: U.S. Pat. No. 11,464,443 B2.

We claim:

1. A machine-learning (ML) based motion analysis and training method, comprising:
acquiring data of measurements of kinematics of a user performing a task with his/her hands or arms;
analyzing the data of measurements based on a machine-learning (ML) model to evaluate patterns of hand/arm movements of the user; and
providing feedback and/or advice based on results of the analysis for improvement of skills of the hand/arm motions of the user.

2. The ML based motion analysis and training method of claim 1, wherein the acquiring data of measurements comprises measuring and collecting data of kinematic and/or temporal features of the movements through inertial measurements.

3. The ML based motion analysis and training method of claim 1, wherein the providing feedback and/or advice comprises providing instructions/feedback to the user based on the results of the analysis for the user to achieve desired skills of the hand/arm motions, and displaying the instructions/feedback to the user in forms of text, audio, images, or videos.

4. The ML based motion analysis and training method of claim 1, wherein the analyzing the data of measurements comprises data preprocessing configured to prepare raw data for analysis, feature engineering configured to create relevant features from the raw data for training the ML model, model training configured to train the ML model based on the preprocessed data, model evaluating configured to assess performance of the trained model based on predetermined evaluation metrics, hyperparameter tuning configured to fine-tune the hyperparameters of the ML model to improve its performance, model validating, and model testing.

5. The ML based motion analysis and training method of claim 4, wherein the data preprocessing comprises one or some or all of the steps including data cleaning, handling missing values, data transforming, and feature extracting.

6. The ML based motion analysis and training method of claim 4, wherein the feature engineering comprises one or some or all of the steps including dimensionality reduction, scaling, encoding categorical variables, and creating new features.

7. The ML based motion analysis and training method of claim 4, wherein the model training comprises feeding the data into the selected model and adjusting the model parameters to learn patterns from the data.

8. The ML based motion analysis and training method of claim 4, wherein in the step of model evaluating, the evaluation metrics comprise accuracy, precision, recall, F1-score, or area under the ROC curve (AUC-ROC).

9. The ML based motion analysis and training method of claim 4, wherein the hyperparameters of the step of hyperparameter tuning are settings that are not learned during the model training, including one or more of learning rate, regularization strength, and a number of hidden layers in a neural network.

10. The ML based motion analysis and training method of claim 4, wherein the data are split into a training set, a validation set, and a testing set to evaluate the model's performance on unseen data.

11. The ML based motion analysis and training method of claim 4, wherein the model training is based on training a support vector machine (SVM) model with the preprocessed data.

12. The ML based motion analysis and training method of claim 4, wherein the feature engineering comprises automatic segmentation by determining thresholds of the data.

13. The ML based motion analysis and training method of claim 12, wherein the automatic segmentation is performed by applying ruptures library for detecting changepoints of the data.

14. The ML based motion analysis and training method of claim 13, wherein the applying the ruptures library comprises applying a Pelt method.

15. The ML based motion analysis and training method of claim 13, wherein the applying the ruptures library comprises applying a Window method.

16. A computer program product, comprising:
a non-transitory computer-executable storage device having computer readable program instructions embodied thereon that when executed by a computer cause the computer to perform a machine-learning (ML) based motion analysis and training method, the computer-executable program instruction comprising:
acquiring data of measurements of kinematics of a user performing a task with his/her hand or arm;
analyzing the data of measurements based on a machine-learning (ML) model to evaluate patterns of hand/arm movements of the user; and
providing feedback and/or advice based on results of the analysis for improvement of skills of the hand/arm motions of the user.

17. A machine-learning (ML) based motion analysis and training system, comprising:
a data acquisition module configured for collecting data of measurement of kinematics of a user;
a skill analysis module configured for wirelessly communicating with the data acquisition module and analyzing the data collected based on a machine-learning (ML) model to evaluate patterns of hand/arm movements of the user; and
an instruction module configured for providing feedback and/or advice for improvement of skills of the hand/arm motions of the user.

18. The ML based motion analysis and training system of claim 17, wherein the skill analysis module is configured to train a support vector machine (SVM) model with the data of collected.

19. The ML based motion analysis and training system of claim 17, wherein the skill analysis module is configured to preprocess data to prepare raw data for analysis.

20. The ML based motion analysis and training system of claim 17, wherein the skill analysis module is configured to perform feature engineering to create relevant features from the raw data for training the ML model.
